(19) United States
(12) Patent Application Publication
(10) Pub. No.: US 2024/0261573 A1
(43) Pub. Date: Aug. 8, 2024

(54) LATERAL EPIDURAL STIMULATION TO AFFECT KIDNEY FUNCTION

(71) Applicant: Medtronic, Inc., Minneapolis, MN (US)

(72) Inventors: Lilian Kornet, Eijsden (NL); Steven M. Goetz, North Oaks, MN (US); Andrew J. Cleland, Macquarie Park (AU)

(21) Appl. No.: 18/427,933

(22) Filed: Jan. 31, 2024

Related U.S. Application Data
(60) Provisional application No. 63/443,213, filed on Feb. 3, 2023.

(51) Int. Cl. A61N 1/36

(52) U.S. Cl. CPC A61N 1/36062; A61N 1/36139; A61N 1/36146

(57) ABSTRACT

Systems, devices, and methods for affecting a patient's kidney function by activating the reno-renal reflex via electrical stimulation of preganglionic dorsal root fibers using one or more electrodes placed proximate preganglionic dorsal root fibers of one or more dorsal roots at the T10 to L1 vertebrae of the patient's spinal cord.

### RELATED APPLICATIONS

This application claims the benefit of U.S. Provisional Application Ser. No. 63/443,213, filed Feb. 3, 2023, the entire contents of each of which are incorporated herein by reference.

The present disclosure is directed to influencing organ function, and more specifically, systems, devices, and methods to influence the functioning of the kidneys through stimulating nerve fibers.

Nerve tissue contains both efferent fibers and afferent fibers. Electrical signals propagate from the central nervous system to tissue/organs along efferent fibers while electrical signals propagate from tissues/organs to the central nervous system along afferent fibers. Applying electrical signals to nerve fibers (e.g., afferent fibers) of the dorsal root ganglion (DRG) can be used to innervate targeted organ functions to alleviate patient discomfort or as a part of addressing other patient conditions or symptoms. However, dorsal root ganglion stimulation may be a challenging procedure and physicians that perform such procedure may undergo advanced training. Additionally, placing a lead through the foramen can be painful, multiple leads may be required for effective DRG stimulation, the leads used for DRG stimulation may be susceptible to fracture, and foraminal stenosis can be exacerbated by DRG stimulation.

### SUMMARY

Embodiments described herein are directed to illustrative systems, devices, and methods to promote diuresis by activating neural pathways using neurostimulation, e.g., for managing heart failure or hypertension in patients. Generally, the illustrative systems, devices, and may be described as activating renal afferent nerves by delivering stimulation to preganglionic dorsal root fibers using electrodes.

One illustrative system may include at least one electrode to deliver electrical stimulation to preganglionic dorsal root fibers of a patient to activate renal afferent nerves, thereby innervating at least one kidney of the patient, and a computing apparatus. The computing apparatus may include a processor and the computing apparatus may be operably coupled to the at least one electrode. Further, the computing apparatus may be configured to control the electrical stimulation delivered using the at least one electrode to the preganglionic dorsal root fibers to inhibit activation of renal efferent nerves, thereby innervating the at least one kidney to promote diuresis.

One illustrative method may include delivering electrical stimulation using at least one electrode to preganglionic dorsal root fibers of a patient to activate renal afferent nerves, thereby innervating at least one kidney of the patient. The illustrative method may further include controlling the electrical stimulation delivered using the at least one electrode to the preganglionic dorsal root fibers to inhibit activation of renal efferent nerves, thereby innervating the at least one kidney to promote diuresis.

The above summary is not intended to describe each disclosed embodiment or every implementation of the present disclosure. The figures and the detailed description below more particularly exemplify illustrative embodiments.

### BRIEF DESCRIPTION OF DRAWINGS

The discussion below refers to the following figures, wherein the same reference number may be used to identify the similar/same component in multiple figures. However, the use of a number to refer to a component in a given figure is not intended to limit the component in another figure labeled with the same number. The figures are not necessarily to scale.

FIG. 1A is a schematic diagram of the anatomical pathways of renal efferent and afferent neurons.

FIG. 1B is a cross-sectional axial view of the anatomy of a vertebra of a human spinal column.

FIG. 2 is a diagram of an illustrative implantable system configured to deliver stimulation of the preganglionic dorsal root fibers.

FIGS. 3A and 3B are diagrammatic views of implantable leads positioned in the spinal epidural space, e.g., that could be used in the system of FIG. 2.

FIG. 4 is a block diagram of the implantable system of FIGS. 2-3.

FIG. 5A is a block diagram of an illustrative method of preganglionic dorsal root fiber stimulation to activate a patient's reno-renal reflex, e.g., that may be performed or executed by the system of FIGS. 2-4.

FIG. 5B is a block diagram of an illustrative method of preganglionic dorsal root fiber stimulation to influence a decrease in blood pressure, e.g., that may be performed or executed by the system of FIGS. 2-4.

FIG. 6 is a block diagram of an illustrative method of stimulating preganglionic dorsal root fibers in response to one or more physiological parameters, e.g., that may be performed or executed by the system of FIGS. 2-4.

FIG. 7 is a block diagram of an illustrative method of adjusting one or more parameters of electrical stimulation to preganglionic dorsal root fibers in response to data, e.g., that may be performed or executed by the system of FIGS. 2-4.

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilize, and structural changes may be made without departing from (e.g., still falling within) the scope of the present disclosure.

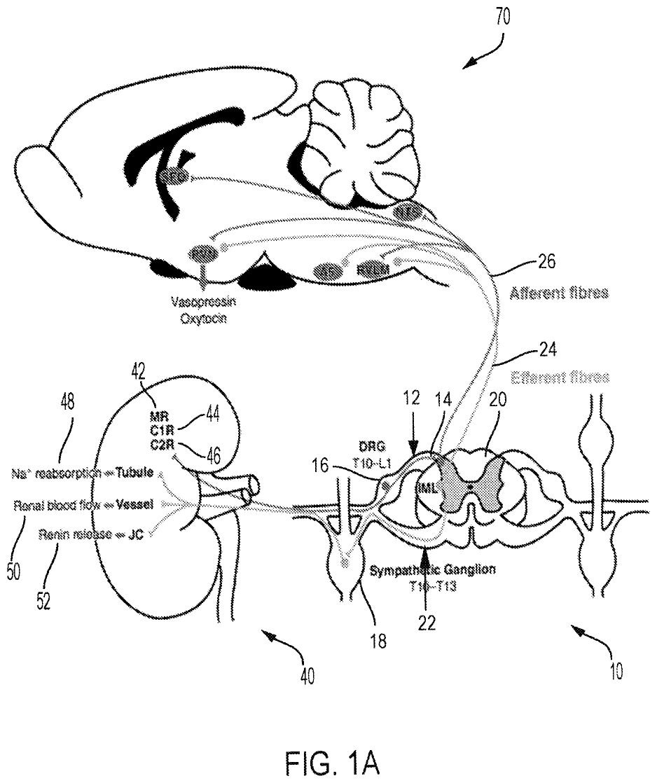

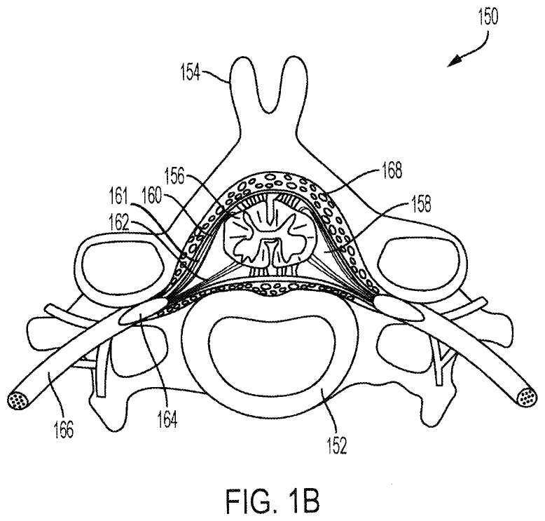

Illustrative systems, devices, and methods shall be described with reference to FIGS. 1-7. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures or described herein. Further, it will be recognized that the embodiments described herein may include elements that are not necessarily shown to scale. Still further, it will be recognized that timing of the processes and the size and shape of various elements herein may be modified but still fall within the scope of the present disclosure.

During heart failure episodes (e.g., diastolic heart failure or systolic heart failure), such as acute decompensated heart failure (ADHF), kidney flow and function are overactivated, which may lead to less diuresis and a higher blood volume, or volume overload. Further, an already weakened heart may struggle to effectively process an increased blood volume, which may lead to congestion and fluid movement into nearby organs, potentially causing dyspnea and edema of one or more organs.

Acute decompensated heart failure consumes about 60% of the heart failure healthcare resources ($36 B annually) in developed countries, with costs mainly attributed to hospitalizations. Mortality and readmission rates within 60 and 90 days of discharge for patients hospitalized with HF approach 15% and 30%, respectively 1. As atrial fibrillation begets atrial fibrillation, ADHF can be described as anticipating chronic decompensated HF (CHDF) and CDHF can be described as anticipating ADHF. Complications from ADHF may include, for example, hemodynamic failure, arrhythmias (e.g., atrial fibrillation, ventricular tachycardia, and ventricular fibrillation), organ damage due to oxygen shortage (e.g., liver, kidney, and brain), acute respiratory distress, cardiac valve dysfunction, leg venous stasis, and ulcers.

Unfortunately, certain methods to increase diuresis may grow ineffective or even hazardous when used for extended periods of time. For example, a patient with congestive heart failure may frequently have “loop diuretics” administered to increase diuresis. Loop diuretics inhibit the Na+/K+/2Cl− co-transport in the ascending part of Henle's loop in nephrons, by which the sodium concentration in the blood decreases and reduces the reabsorption of water, which may result in increased urine production. However, with chronic use, loop diuretics may be toxic for kidneys and can lead to structural changes of a kidney such as hypertrophy of the epithelial cells in the distal tubules, which may enhance distal reabsorption of sodium and limit sodium excretion and diuresis. The need to increase the dosage of loop diuretic medication over time may be exacerbated because the use of loop diuretics lowers the dose-response curve. In addition, loop diuretics might interact with heart failure medication and may be less well absorbed if the stomach lining is damaged, as often occurs in heart failure patients, making loop diuretics less effective. That is to say, oral medications may be described as having limited efficiency in heart failure patients due to impaired absorption in the stomach and gut. Efficacy of oral medications may be further limited in heart failure patients by interactions with other heart failure medications and nonsteroidal anti-inflammatory medication. It is estimated that 40% of hospitalized heart failure patients are discharged without resolved congestion due to diuretic resistance. Moreover, the chronic intake of medication can negatively affect a patient's psychological health, for example by being a continual reminder of the patient's health concerns. Further, patient compliance in self-administration of loop diuretics can be an issue.

Illustrative systems, devices, and methods described herein may utilize a negative feedback loop referred to as the “reno-renal reflex.” In healthy patients, the reno-renal reflex is activated by the renal efferent nerves, which stimulate water retention thereby increasing blood volume. Activation of the reno-renal reflex promotes a continuous retention of water by the kidneys. The reflex additionally involves activation of the afferent nerves to inhibit the renal efferent nerves thereby promoting diuresis. However, in patients with congestive heart failure or acute decompensated heart failure, the activation of the renal afferent nerves may be impaired. Therefore, efferent nerves, and resultingly, water reabsorption, may not be adequately suppressed in patients with congestive heart failure or acute decompensated heart failure.

Illustrative systems, devices, and methods described herein may be described as activating the renal nerves (e.g., afferent nerves) through electrical stimulation of the preganglionic dorsal root fibers of the corresponding dorsal roots of such renal nerves to mimic the electrical activation signals of such renal nerves. The electrical stimulation may be delivered to the preganglionic dorsal root fibers, for example, by one or more electrodes positioned (e.g., one or more implanted in the lateral epidural space inside the patient and located on the outside of the patient proximate the spine) proximate to the preganglionic dorsal root fibers. Stimulating preganglionic dorsal root fibers using electrodes implanted in the lateral epidural space as opposed to electrically stimulating dorsal root ganglions may afford easier placement of electrodes. In other words, it may be one or more of less complicated and quicker to position electrodes proximate the preganglionic dorsal root fibers for stimulation thereof than positioning electrodes proximate the dorsal root ganglions to stimulate the dorsal root ganglions.

Further, stimulation of unintended nerves (e.g., overstimulation) may be challenging when delivering stimulation to the preganglionic dorsal root fibers. For example, nearby non-target fibers may be stimulated in addition to stimulation of the preganglionic dorsal root fibers. Additionally, cerebrospinal fluid in the lateral epidural space may propagate stimulation (e.g., current, signals, etc.) from stimulation delivered to the preganglionic dorsal root fibers to unintended nerves (e.g., non-target fibers of other nerves). Stimulating unintended nerves can cause side effects, such as, for example, shivering, muscle contractions, or pain. The illustrative systems, devices, and methods described herein may be described as providing adjustments, feedback loops, controls, initial parameters, and electrode placements that limit (e.g., avoid, prevent, reduce, etc.) stimulation of unintended nerves (e.g., overstimulation).

A schematic diagram of the anatomical pathways of renal efferent and afferent neurons is shown in FIG. 1A. As shown, the anatomical pathways include vertebral neural structures 50, shown as an axial cross-section. The vertebral neural structures 50 may include a dorsal root 12 (including, e.g., preganglionic dorsal root fibers 14), a dorsal root ganglion 16, a sympathetic ganglion 18, a spinal cord 20, a ventral root 22.

Renal afferent nerve fibers 26 may be described as traversing, or extending through, the vertebral neural structures 50, including, for example the dorsal root 12, the preganglionic dorsal root fibers 14, and the dorsal root ganglion 16. The renal afferent nerve fibers 26 may be further described as travelling, or extending, peripherally, or outwardly, from the vertebral neural structures 50 to one or both kidneys (e.g., kidney 40). The renal afferent nerve fibers 26 may be still further described as travelling, or extending, up the spinal cord 20 (i.e., in the cephalad direction, or headward) from the vertebral neural structures 50 to a brain 70.

The renal efferent nerve fibers 24 may be described as traversing, or extending through, the vertebral neural structures 50, including, for example, the sympathetic ganglion 18 and the ventral root 22. Like the renal afferent nerve fibers 26, the renal efferent nerve fibers 24 may be described as travelling, or extending, peripherally from the vertebral neural structures 50 to one or both kidneys (e.g., the kidney 40) and up the spinal cord 20 from the vertebral neural structures 50 to the brain 70.

In general, the renal afferent nerve fibers 26 may be described as carrying, or transmitting, sensory signals (e.g., from mechanoreceptors 42 and chemoreceptors 44, 46 sensing changes in the kidney 40) from the kidney 40 to the brain 70. The renal efferent nerve fibers 24 may generally be described as carrying, or transmitting, control signals (e.g., affecting one or more of sodium reabsorption 48, renal blood flow 50, and renin release 52) from the brain 70 to the kidney 40. As described herein, the illustrative systems, devices, and methods may be configured to deliver stimulation to the preganglionic dorsal root fibers 14 using one or more electrodes positioned proximate the preganglionic dorsal root fibers 14 to activate renal nerve fibers (e.g., renal afferent nerve fibers 26). Stimulation of neurons of renal afferent nerve fibers 26 leads to inhibition of neurons of renal efferent nerve fibers 24 inhibition and, subsequently, to increased diuresis and lowered blood volume. Renal afferent neurons, traverse the preganglionic dorsal root fibers at the T10-L1 level. Therefore, when using preganglionic dorsal root fiber stimulation to influence kidney function, the electrodes and stimulation may be targeted at the preganglionic dorsal root fibers of the dorsal roots of the T10-L1 vertebrae of the spine.

A cross-sectional axial view of the anatomy of a vertebra 150 of a human spinal column is depicted in FIG. 1B. As shown, the vertebra 150 includes a vertebral body 152 and a bony ring that includes the spinous process 154. The spinal column 156 is surrounded by cerebrospinal fluid 158 and is positioned between the vertebral body 152 and bony ring. A dorsal root 160 and ventral root 162 extend from the spinal column 156 toward the vertebral body 152 on both the right and left sides. The dorsal root ganglion 164 is positioned at the distal end of the dorsal root 160 and contains the neuron cell bodies of the nerve fibers conveyed by the root. The preganglionic dorsal root fibers 161 extend from the proximal end of the dorsal root 160 toward the dorsal root ganglion 164. The dorsal root 160 and the ventral root 162 join to form a mixed spinal nerve 166. The space between the bony ring and the spinal column 156/dorsal root 160 is called the epidural space 168. As described herein, the illustrative systems, devices, and methods described herein may be configured to deliver stimulation the preganglionic dorsal root fibers 161.

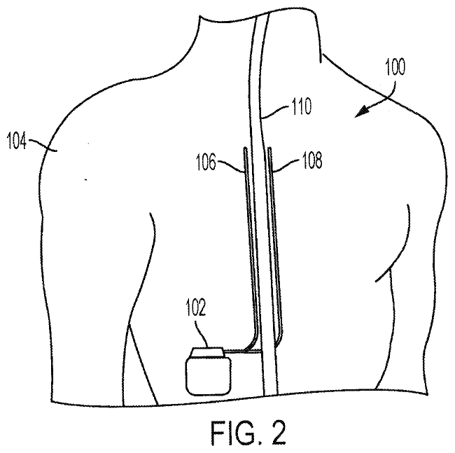

For example, electrical stimulation of the patient's preganglionic dorsal root fibers may be controlled and administered by an illustrative implantable system 100 implanted in a patient 104 as depicted in FIG. 2. The implantable system 100 may include a device 102 and two leads 106, 108 electrically coupled to the device 102. The implantable system 100 may be configured as an electrical stimulator that generates and delivers electrical stimulation to the patient 104 via one or more electrodes of the leads 106, 108. Although the implantable system 100 shown in FIG. 2 includes two leads 106, 108, it is to be understood that illustrative systems, devices, and methods described herein may include a single lead or more than two leads (e.g., one lead, two or more leads, three or more leads, a plurality of leads, etc.).

The implantable system 100 may be a chronic system configured to remain implanted within the patient 104 on the order of days to years. Conversely, the implantable system 100 may be a temporary or trial system used to screen or evaluate the efficacy of electrical stimulation. The leads 106, 108 may be positioned in the lateral epidural space of the patient's 104 spinal column 110 to deliver stimulation to the preganglionic dorsal root fibers as will be described further herein. The leads 106, 108 may be positioned such that the electrodes are positioned a suitable distance from the patient's spinal midline, or spinal centerline. Suitable distances from the patient's spinal midline may include, for example, greater than 2 millimeters (mm), greater than 3 mm, greater than 4 mm, or greater than 6 mm, and/or less than 6 mm, less than 5 mm, less than 3 mm, or less than 2 mm. For example, a suitable distance from the patient's spinal midline may be 4 mm or between 3 mm and 5 mm.

Additionally, although the system 100 is shown in FIG. 2 as being fully implanted, in some configurations, the device 102 may be an external device coupled to percutaneously implanted leads 106, 108. More specifically, the leads 106, 108 may each be a percutaneous stimulation lead used to deliver various electrical stimulations. In some embodiments, a percutaneous stimulation lead may have cylindrical electrodes arrayed longitudinally along a lead body. Electrical stimulation may be delivered by applying a series of electrical stimulation pulses to a selected set of the electrodes.

In addition to electrical stimulation, the implantable system 100 may also be configured to generate and deliver control pulses configured to elicit (e.g., trigger or initiate) evoked compound action potential (ECAP) signals. The control pulses may not stimulate the preganglionic dorsal root fibers to activate renal afferent nerves innervating at least one of the patient's kidneys to increase diuresis as part of the reno-renal reflex. In one or more embodiments, some of the electrodes can be configured to sense an ECAP in response to the stimulation pulses. The stimulation pulses may or may not stimulate the preganglionic dorsal root fibers to activate renal afferent nerves innervating at least one of the patient's kidneys to increase diuresis as part of the reno-renal reflex, and the sensed ECAP response may facilitate measuring the efficacy of the applied pulses.

The electrodes may additionally or alternatively be configured to sense electromyographic (EMG) data, for example, to measure side effects such as muscle contractions or shivering. EMG data may also be measured with external (e.g., skin-worn) electrodes such as, e.g., in a clinical programming session or during an operating room procedure to implant the implantable system 100.

Additionally or alternatively to the electrodes for stimulation of the preganglionic dorsal root fibers, one or more additional electrodes (e.g., the additional electrodes are not the same electrodes use for stimulation) for sensing may be operably coupled to the implantable system 100 (e.g., via the leads 106, 108 or additional leads) and positioned (e.g., implanted in the tissue of the patient or placed on the skin of the patient) to sense the ECAP or EMG to the stimulation pulses. The one or more additional electrodes for sensing may be positioned in any suitable location. Suitable sensing electrode locations may include, for example, proximate the same preganglionic dorsal root fiber as a corresponding stimulating electrode, proximate postganglionic nerve fiber on the same nerve as a corresponding stimulating electrode, or proximate the patent's spinal column (e.g., upstream or downstream of one or more corresponding electrodes for stimulating), or in other tissues known to exhibit stimulation side effects. As described above, any electrode, including the electrodes used for stimulation, may operate as a sensing electrode.

In some embodiments, each of the leads 106, 108 may include a lead body, which may be defined as an elongated cylindrical tube, and which may be implanted using relatively non-intrusive surgical procedures. The electrodes on such leads 106, 108 may be cylindrical rings arrayed longitudinally down the length of the leads 106, 108. Two or more electrodes may be configured to provide stimulation, for example, by being electrically coupled to respective anodic and cathodic outputs of a pulse generator of the device 102. For an ECAP process, two or more different electrode segments (e.g., at a distal end of the lead) segments may be electrically coupled to sense the evoked response, with one electrode being coupled as an anode and the other as a cathode. The leads 106, 108 may have any number of electrodes (e.g., up to or more than four) to allow for customizing the location of the stimulation and sensing. This customization can be performed electronically by the selection of different subsets of the electrodes for each function, for example, using a switching circuit.

Percutaneous, epidural, cylindrical electrodes for spinal cord stimulation may provide less optimal targeting of the stimulation field to the target neural structures. For preganglionic dorsal root stimulation, the leads 106, 108 may be positioned in an epidural space near the dorsal column of the patient's spine. In this configuration, one side of each lead 106, 108 may face the nerve tissue of the spinal cord. The one or more parts, or portions, of the stimulation electrodes facing away from the spinal cord may be deliver (e.g., emit) stimulation into non-target tissue, and the part, or portion, of the sensing electrodes facing away from the spinal cord may receive electromagnetic impulses unrelated to the preganglionic dorsal root fibers stimulation.

Accordingly, in some embodiments described herein, directional subcutaneous lead electrodes may be configured to sense and emit electromagnetic fields over a first partial circumference of the lead body, such that there is no respective sensing and emission along a second partial circumference at the same longitudinal location. In such embodiments, the second partial circumference is different than the first partial circumference (e.g., the partial circumferences may be non-overlapping). This may be achieved by segmenting the electrodes at each longitudinal location, or by covering parts of fully circumferential electrodes (e.g., ring electrodes) with an electrical insulator that suppresses emission/sensing along the covered portions. The segmented electrodes may be controlled to steer the resulting fields to stimulate preganglionic dorsal root fibers (e.g., cathode towards the target structure and anode away) or to adjust stimulation laterally (e.g., more laterally, less laterally, closer to the patient's spinal midline or farther from the patient's spinal midline), such as to account for non-optimal lead positioning. Further steering may be achieved, for example, by staggering directional electrodes with nondirectional electrodes to save contacts and extend the lead to cover preganglionic dorsal root fibers of more dorsal roots. Similar to any of the lead electrodes, the segments of the directional electrodes may be individually programmable using different parameters (e.g., ping, synchronous, asynchronous, continuous, or discontinuous) to control the depth of field for the stimulation.

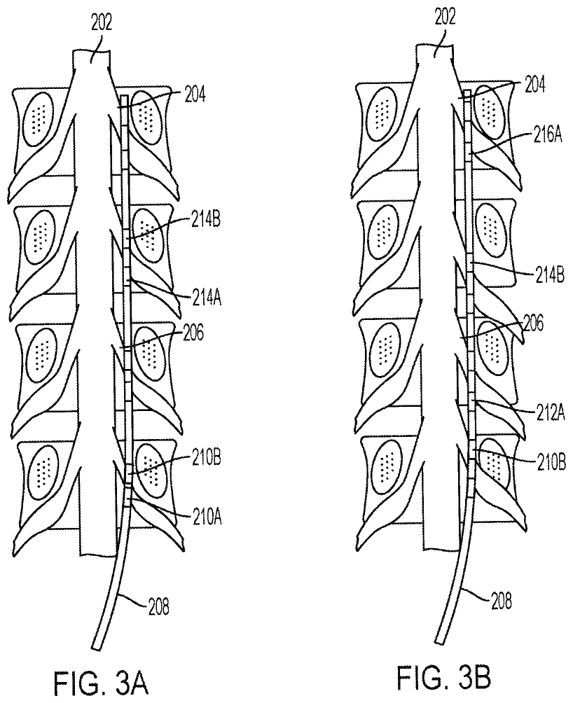

Two illustrative lead configurations used for delivering preganglionic dorsal root fibers stimulation as described herein are depicted in FIGS. 3A-3B. As shown, leads 208 of FIGS. 3A-3B may be positioned (e.g., implanted in tissue of the patient) along a patient's spinal column 202 to position electrodes (some of which are labeled) supported by the leads 208 proximate the target preganglionic dorsal root fibers (e.g., preganglionic dorsal root fibers 204, 206). The electrodes may be configured as electrode pairs, as shown in FIGS. 3A and 3B. While each lead 208 is shown with four electrode pairs, each lead may have fewer, or more, electrodes. For example, a lead may have four to eight electrodes. For another example, a lead may have a single electrode or two electrodes. In some embodiments, each of the leads 208 may have a different number of electrodes.

The leads 208 may be positioned to target (i.e., positioned with electrodes proximate to) the preganglionic dorsal root fibers of either one of the left or right dorsal roots, as shown in FIGS. 3A and 3B. In other embodiments, the leads 208 may be positioned to target the preganglionic dorsal root fibers of both the left and right dorsal roots (see FIG. 2). The leads 208 may be positioned to target the preganglionic dorsal root fibers of multiple levels of the patient's spinal column 202 (e.g., four different levels, as shown in FIGS. 3A and 3B). In other embodiments, the leads 208 may be positioned to target preganglionic dorsal root fibers of one or more levels of the patient's spinal column 202. For example, the leads 208 may be positioned to target the preganglionic dorsal root fibers of both the left and right dorsal roots of either of the patient's T11 or T12 vertebrae or either of the T10 or T11 vertebrae. For yet another example, the leads 208 may be positioned to target the preganglionic dorsal root fibers of both the left and right dorsal roots of the patient's T11 and T12 vertebrae. In still another example, a lead 208 may be positioned to target the preganglionic dorsal root fibers of the left dorsal roots of the T11 vertebra and another lead 208 may be positioned to target the preganglionic dorsal root fibers of the right dorsal roots of the T12 vertebra.

Further, the two different lead configurations of FIGS. 3A-3B have, or define, different spacing configurations of the electrodes along the leads. In certain embodiments, the spacing between two electrodes in an electrode pair may be varied as shown in FIG. 3A, in which one lead 208 is shown extending over four neural structures (here, preganglionic dorsal root fibers 204 of four dorsal roots) of four sequential levels of a patient's spinal column 202. As set forth above, the lead 208 could cover preganglionic dorsal root fibers 204 of one or more dorsal root extending from a proximal end of the dorsal nerve roots toward the dorsal root ganglions. The preganglionic dorsal root fibers 204 of each dorsal root are covered by a pair of electrodes (e.g., 210A and 210B). To improve targeting stimulation at desired preganglionic dorsal root fibers 204 of each dorsal root, the spacing within each electrode pair can be varied. For example, electrodes 210A and 210B are spaced a first distance apart (measured from the center of electrode 210A to the center of electrode 210B) whereas electrodes 214A and 214B are spaced a second distance apart (measured from the center of electrode 214A to the center of electrode 214B) where the first distance and the second distance are not equal (e.g., the first distance may be greater than or equal to the second distance, the second distance may be greater than or equal to the first distance). Each of the first and second distances may be between about 3 millimeters (mm) to about 14 mm. In at least one embodiment, one or both of the first and second distances is 3 mm. Alternatively, the distances may be configured as a ratio of a distance between electrodes per the length of the lead or per number of electrodes present. The spacing may be determined by the size (e.g., width) of the targeted preganglionic dorsal root fibers 204. The spacing may also be controlled by placement of electrodes of different sizes. Electrodes may have a diameter in a range of sizes such as less than 1 mm to 1.5 mm, or 1 mm to 1.3 mm or even a diameter of 1 mm. The electrode heights may be between about 1 mm and about 8 mm, and some illustrative embodiments, electrode heights may be 4 mm.

Additionally or alternatively, heights of electrodes may vary between electrodes along the length of the lead, such as, for example, an electrode pair having a taller electrode and a shorter electrode (i.e., shorter in the axial direction of the patient's spinal column than the taller electrode). In such an example, the taller electrode may be 2 mm and the shorter electrode may be 4 mm. In another example, a lead may have a longer, or taller, electrode in the most caudal and/or the most cephalad position along the length of the lead. In such an example, the longer electrode(s) may be 4 mm and the other electrodes along the length of the lead may be 2 mm.

Spacing variation between electrode pairs (i.e., interelectrode pair spacing) is illustrated in FIG. 3B. The spacing variations of FIGS. 3A and 3B may be used in any combination. For interelectrode pair spacing, the space between the second electrode of a first pair and a first electrode of a subsequent pair is varied. As shown, the neural target structures (e.g., preganglionic dorsal root fibers) of the spinal column 202 are not evenly spaced apart. Therefore, electrodes 210B and 212A are spaced apart by a third distance, and electrodes 214B and 216A are spaced apart by a fourth distance, where the third distance and the fourth distance are not equal (e.g., the third distance may be greater than or equal to the fourth distance, the fourth distance may be greater than or equal to the third distance). Each of the first and second distances may be between about 1 mm to about 35 mm depending on the patient and location of the target preganglionic dorsal root fibers. For example, one or both of the first and second distances may be greater than or equal to 1 mm, greater than or equal to 5 mm, greater than or equal to 15 mm, greater than or equal to 20 mm, greater than or equal to 30 mm, or greater than or equal to 35 mm, and/or less than or equal to 40 mm, less than or equal to 25 mm, less than or equal to 15 mm, less than or equal to 10 mm, or less than or equal to 1 mm. In at least one embodiment, one or both of the first and second distances is 3 mm. There is variability in distances between target structures of different vertebral levels, for example with respect to a patient's height or degeneration of the intervertebral discs. In addition, in the cervical region, the distances may be smaller, closer to 3 mm, and larger in the lumbar region, up to about 35 mm. Additionally or alternatively, the distances may be configured as a ratio of a distance between electrode pairs per the length of the lead or per number of electrodes present.

The varying distances between the electrode pairs may be due to varying distances between neural structures (e.g., preganglionic dorsal root fibers of sequential dorsal roots); due to different lengths of cervical, thoracic, and lumbar levels; or due to differences between patients, such as differences in height between patients. The sizing can be measured by fluoroscopy for customization for a patient, for example by measuring pedicle spacing. In some embodiments, the spacing may be 3, 3, 2 spacing for an example eight-contact lead, which could also anticipate migration by providing redundancy in ⅔ of the pairs. In other leads, two four-contact electrode clusters may protect against migration. As discussed above, the electrodes may be individually controlled, or segments of a directional electrode may be individually controlled, for example, to configure multi-polar leads as mono-polar leads. As set forth, a lead may also include more than eight contacts, which may provide additional spacing options. The electrodes are controlled by a control unit as part of an implantable system discussed further below.

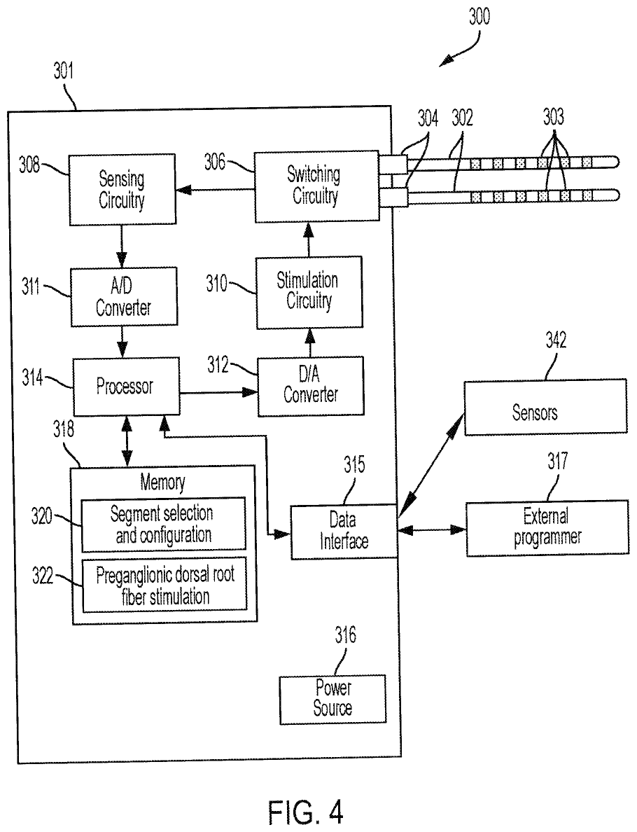

A block diagram of an implantable system 300 is shown in FIG. 4. As shown, the system 300 includes a control unit 301, which may be described as a self-contained unit that may be implanted within the patient or located externally, and one or more leads 302 that are operatively coupled to the control unit 301 via connectors 304. The control unit 301 may be described as a computing apparatus and may include any componentry configured to deliver preganglionic dorsal root fiber according to the methods and processes described herein. For example, the control unit 301 includes, among other things, switching circuitry 306 that selectively couples electrodes 303 (and segments thereof, if so configured) of the leads 302 to individual circuit elements within the control unit 301. Further, the control unit 301 may include sensing circuitry 308, operatively coupled to the electrodes 303 via the switching circuitry 306 to receive signals such, e.g., as ECAP measurements or EMG measurements. Additionally or alternatively, the electrodes 303 may be coupled to stimulation circuitry 310 via the switching circuitry 306 to deliver electrical stimulation (e.g., signals, pulse waveforms, etc.) to target tissue via the electrodes 303.

The switching circuitry 306, sensing circuitry 308, and stimulation circuitry 310 may include analog processing circuitry such as preamplifiers, amplifiers, and filters, and any other electrical or electronic component so as to perform the methods and processes described herein. As the control unit 301 may use digital signal processing, the control unit 301 may utilize an analog-to-digital converter 311 and/or a digital-to-analog converter 312. These facilitate digital signal processing via processors 314 (or one or more processors). The processor 314 may include any combination of central processing units, co-processors, digital signal processors, application specific integrated circuits, etc. The processor 314 is coupled to memory 318, which may include any combination of volatile memory (e.g., random access memory) and non-volatile memory (e.g., firmware, flash memory).

The system 300 may further include sensors 342 such as pressure sensors, accelerometers, flow sensors, blood chemistry sensors, activity sensors, or other physiological sensors known for use with implantable medical devices. The sensors 342 may be coupled to the control unit 301 via the data interface 315 that may provide sensor signals to the processor 314. The sensor signals may be used by the processor 314 for detecting physiological events or conditions of a patient. For example, the control unit 301 may monitor various physiological parameters (e.g., a patient's creatinine level, blood urea nitrogen level, respiration rate, abdominal fluid content or fluid content in thoracic tissue, pulmonary wedge pressure). Monitored signals may be used, for example, to determine whether to deliver, adjust, terminate, or initiate preganglionic dorsal root fibers stimulation.

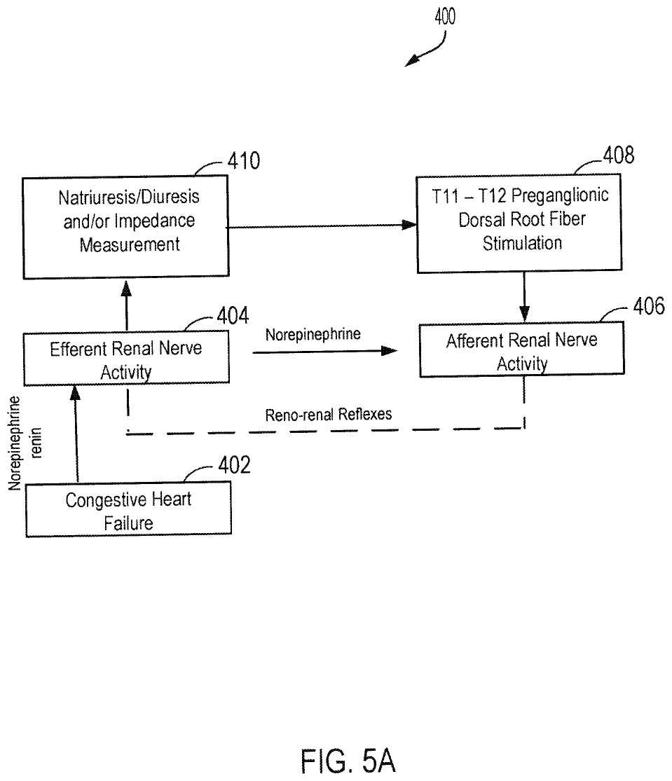

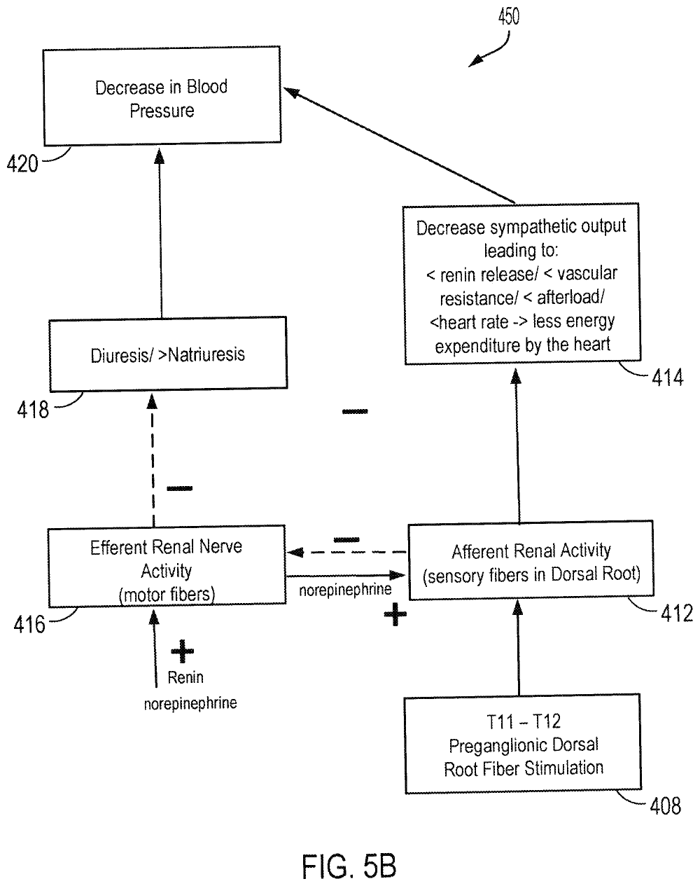

The processor 314 operates in response to instructions stored in the memory 318. Those instructions may include a segment selection and configuration functionality 320 that assists in setup of leads having one or more segmented electrodes. The instructions may also include preganglionic dorsal root fibers stimulation functionality 322 for controlling stimulation to preganglionic dorsal root fibers as described further herein with reference to FIGS. 5A-7. The memory 318 may further store a variety of programmed-in operating mode and parameter values that are used by the processor 314. The memory 318 may also be used for storing data compiled from sensed signals and/or relating to device operating history (e.g., abdominal impedance for use in delivering, adjusting, controlling, initiating, and/or terminating stimulation) and/or for communicating such data outside of the patient. Examples of data compiled from sensed signals and/or relating to device operating history may include, for example, parameters of electrical stimulation (e.g., frequency, sinusoidal current, voltage, pulse width, tonic or burst stimulation, uni- or multi-lateral stimulation, balance of multi-lateral stimulation, on/off cycle timing), stimulation response data (e.g., EMG data or ECAP data, impedance sensor measurements, blood pressure sensor measurements), side effect response data (e.g., EMG data, ECAP data, sensor measurements of electrolyte levels, etc.).

The system 300 may further includes an external programmer 317. The control unit 301 may be able to be programmed or controlled via the external programmer 317. The external programmer 317 links with the data interface 315 of the device. The data interface 315 may facilitate communications via any combination of wireless media, wired media, optical media, etc. The external programmer 317 may send control instructions to the control unit 301, add software/firmware to the control unit 301, update software/firmware of the control unit 301, and/or download data gathered by the control unit 301. The control unit 301 may also include a self-contained power source 316 (e.g., battery, capacitors, generator, or converter).

The control unit 301 along with the leads 302 and the electrodes 303 may be used for one or both of steady state stimulation to mimic daily medication intake (e.g., diuretics) and maximal stimulation to mitigate an acute clinical congestive state. This is done by activating the appropriate renal nerves (e.g., afferent nerves) to mimic the activation signals of the renal nerves in the T10-L1 preganglionic dorsal root fibers.

Different fiber types (dorsal column to dorsal root) may have different responses to stimulation at low pulse widths and high amplitudes compared with high pulse widths and low amplitudes (e.g., a strength-duration curve of a fiber type). The difference in responses between different fiber types may be used to selectively stimulate a target, or intended, fiber or to selectively avoid stimulating a non-target, or unintended, fiber. By adjusting between one or both of low pulse width configurations and high amplitude configurations and one or both of high pulse width low amplitude configurations and comparing measured responses at each, pulse width and amplitude may be configured to optimize, or maximize, the patient's stimulation effect response and minimize the patient's side effect response.

In one or more embodiments, ECAP may be used to determine a fiber type or fiber types responding to stimulation pulses. Determining fiber types responding may be used to adjust stimulation parameters to select the target fiber types. For example, the stimulation may be adjusted until the target fiber types respond to the adjusted stimulation. Such embodiments may include measuring a response latency of the ECAP signals to the stimulation pulses. The response latency may be measured, for example, by determining a duration, or time period, between stimulation delivered using a stimulating electrode (e.g., proximate a target preganglionic dorsal root fiber of a target dorsal root) and sensing of the stimulation using a corresponding sensing electrode (e.g., proximate the target preganglionic dorsal root fiber or proximate postganglionic fibers of the dorsal root).

The fiber type(s), or population(s), activated by the stimulation may be determined, or classified, based on the response latency and the known physical distance (e.g., distance of separation) between the stimulating electrode and the sensing electrode. For example, large, myelinated fibers may be described as having a faster signal (e.g., stimulation pulse) propagation, or a lesser response latency, and small unmyelinated fibers may be described as having slower signal (e.g., stimulation pulse) propagation, or a greater response latency. Renal afferent nerve fibers may be described as mainly unmyelinated fibers with a small population of myelinated fibers. Preganglionic dorsal root fibers that run through the T10-L1 are may be described as mainly unmyelinated C-fibers.

The speed of propagation of different fiber types may be used to determine which fiber types are activated by stimulation using certain stimulation parameters. For example, a stimulating electrode (e.g., positioned proximate the preganglionic dorsal root fibers of a dorsal root) may deliver a first series of one or more stimulation pulses and a sensing electrode (e.g., positioned proximate postganglionic fibers of the dorsal root) may thereafter sense the first series of one or more stimulation pulses, and the time delay between the delivery and the sensing of the pulses may be measured, or determined, as a first response latency. If the determined first response latency is not characteristic of afferent renal nerve fibers (e.g., the response latency is low, which may be described as uncharacteristic of afferent renal nerve fibers), one or more parameters of the stimulation may be adjusted. Subsequently, the stimulating electrode may deliver a second series of one or more stimulation pulses, the sensing electrode may thereafter sense the second series of one or more stimulation pulses, and the second response latency may be determined.

If the determined second response latency is more characteristic of afferent renal nerve fibers than the determined first response latency, then the stimulation parameters may be further adjusted (e.g., to further tune the stimulation parameters) or the stimulation parameters may continue to be used (e.g., in stimulating the preganglionic dorsal root fibers to activate renal afferent nerves innervating at least one of the patient's kidneys to increase diuresis as part of the reno-renal reflex). On the other hand, if the determined second response latency is less characteristic of afferent renal nerve fibers than the determined first response latency (e.g., the determined second response latency is lower than the determined first response latency), then the stimulation parameters may be adjusted (e.g., by reversing the previous adjustment or by adjusting different parameters) and the response latency of subsequent series of one or more pulses may be determined.

In one or more embodiments, if the latency response indicates, or suggests, that multiple fiber types are responding to the simulation pulses, the stimulation parameters may be adjusted to at least one of enhance the amplitude, or signal strength, of the ECAP signal(s) characteristic of the target (i.e. desired or intended) fiber types (e.g., the preganglionic dorsal root fibers or the renal afferent nerve fibers) and minimize the amplitude of the ECAP signal(s) characteristic of non-target (i.e., non-desired or non-intended) fiber types. In other words, while adjusting the stimulation parameters may not eliminate response, or stimulation, of non-target fibers, adjusting the stimulation parameters may thereby adjust, or shift, the ratio of responding target fibers to non-target fibers.

The ECAP measurements can be used to adjust the stimulation (e.g., pulse width, amplitude, frequency, and on/off cycle timing) to improve the efficacy of the stimulation, such as by improving selectivity of stimulation (e.g., greater stimulation of target, or intended, fibers and reduced stimulation of non-target, or unintended, fibers). The measurement of the ECAP response allows for, among other things, the manual or automatic adjustment of the stimulation delivered by, or using, the implantable system 100 to compensate for changing conditions over the life of the system and patient. Examples of changing conditions may include, for example, shifting of position or orientation of the lead within the body, or changing physiology of the patient.

The ECAP measurements may additionally or alternatively be used to position the electrodes carried on a lead, such as, e.g., during an operation to implant one or more electrodes. For example, one or more of the ECAP amplitude, morphology (e.g., shape of the signal), and latency may be measured, or monitored, as a stimulating electrode is being positioned during surgery. When the one or more ECAP measurements indicate that the stimulating electrode being positioned is activating the target fiber population (e.g., preganglionic dorsal root fibers of the target dorsal root), positioning of the stimulating electrode may be finalized.

In one or more embodiments, a health care professional placing, or implanting, a lead may advance the lead from a position caudal to a thoracic vertebral level of a target nerve (e.g., using an imaging technique such as fluoroscopy) towards the target thoracic vertebral level. While advancing, ECAP measurements may be received in response to stimulation by an electrode supported, or carried, by the lead, and the ECAP measurements may be presented to the health care professional (e.g., visually on a display). The health care professional may advance the lead to find a position corresponding to, or providing, ECAP measurements that indicate that the stimulating electrode is proximate to and activating the target nerve fibers (e.g., preganglionic dorsal root fibers). Additionally or alternatively, the health care professional placing the lead may adjust medial-lateral positioning of the lead to find a position corresponding to, or providing, ECAP measurements that indicate the stimulating electrode is proximate to and activating the target nerve fibers. For example, the health care professional may so adjust medial-lateral positioning once the vertical (i.e., caudal-cephalad) level is established.

Stimulation parameters used to stimulate the afferent neurons may be controlled or adjusted such that the electrical stimulation avoids stimulating non-targets, or intended, fibers. For example, the stimulation may be described in terms of stimulation waves that have, or define, a tonic form, as a relatively high percentage of the renal afferent neurons (e.g., 48%) may be tonic, which is characteristic for renal nerves. In other words, the stimulation may be described as tonic stimulation to target renal afferent neurons. The tonic stimulation may involve low frequencies between, for example, about 5 hertz and about 120 hertz). In one embodiment, the tonic stimulation may have a frequency of 15 hertz. In one or more embodiments, the tonic stimulation may have a frequency greater than or equal to 5 hertz, greater than or equal to 15 hertz, greater than or equal to 20 hertz, greater than or equal to 35 hertz, greater than or equal to 50 hertz, greater than or equal to 65 hertz, greater than or equal to 80 hertz, or greater than or equal to 95 hertz, and/or less than or equal to 120 hertz, less than or equal to 105 hertz, less than or equal to 90 hertz, less than or equal to 75 hertz, less than or equal to 60 hertz, or less than or equal to 30 hertz. Additionally or alternatively, tonic stimulation may be delivered one or more of continuously with regular periodic pulses, in bursts of pulses with periods of no stimulation, or continuously with irregular timing (e.g., random, pseudo random, or stochastic timing variation) between pulses.

The electrical stimulation delivered to the preganglionic dorsal root fibers may be delivered at a wide variety of different parameters or settings. Such parameters may include, for example, daily or weekly timing, or schedule (e.g., the preganglionic dorsal root fiber stimulation may be delivered for a selected time period). Time periods may include, for example, up to every hour, up to every 4 hours, up to every 8 hours, up to every 10 hours, up to every 18 hours, up to every 24 hours, up to every 30 hours, up to every 48 hours, up to every 4 days, or up to every week. Time periods may include daily, for example, during the daytime. Time periods, or schedules, may additionally or alternatively control stimulation based on the patient's particular activities or schedules, such as based on the patient's circadian rhythm or based on a user-defined, or patient-defined, schedule. Additionally or alternatively, a pre-defined schedule may be defined by a user such as a heath care professional.

Another illustrative parameter for electrical stimulation delivered to the preganglionic dorsal root fibers may include pulses of controlled current, for example with a square waveform. Suitable pulse amplitude currents may include, for example, greater than or equal to 0.025 milliAmps, greater than or equal to 0.04 milliAmps, greater than or equal to 0.09 milliAmps, greater than or equal to 0.12 milliAmps, greater than or equal to 0.2 milliAmps, or greater than or equal to 0.5 milliAmps, greater than or equal to 1 milliAmp, or greater than or equal to 1.5 milliAmps and/or less than or equal to 1.5 milliAmps, less than or equal to 1 milliAmp, less than or equal to 0.5 milliAmps, less than or equal to 0.3 milliAmps, less than or equal to 0.15 milliAmps, less than or equal to 0.05 milliAmps, or less than or equal to 0.025 milliAmps.

Yet another illustrative parameter for electrical stimulation delivered to the preganglionic dorsal root fibers may include a voltage amplitude. Suitable amplitudes may include, for example, greater than or equal to 0.1 volts, greater than or equal to 0.4 volts, greater than or equal to 1.2 volts, greater than or equal to 2 volts, greater than or equal to 3 volts, or greater than or equal to 4 volts, and/or less than or equal to 4 volts, less than or equal to 2.5 volts, less than or equal to 1.5 volts, less than or equal to 0.8 volts, or less than or equal to 0.1 volts. It will be understood in light of this disclosure that voltage-and current-controlled stimulation may alternately be delivered as continuous sinusoidal wave forms or discrete stimulation pulses.

Still another illustrative parameter for electrical stimulation delivered to the preganglionic dorsal root fibers may include frequency of the pulses. Suitable frequencies may include, for example, greater than or equal to 1 hertz, greater than or equal to 5 hertz, greater than or equal to 10 hertz, greater than or equal to 25 hertz, greater than or equal to 40 hertz, or greater than or equal to 50 hertz, and/or less than or equal to 50 hertz, less than or equal to 35 hertz, less than or equal to 15 hertz, less than or equal to 5 hertz, or less than or equal to 1 hertz

Still yet another illustrative parameter for electrical stimulation delivered to the preganglionic dorsal root fibers may include pulse width of each pulse. Suitable pulse widths may include, for example, greater than or equal to 20 microseconds, greater than or equal to 200 microseconds, greater than or equal to 350 microseconds, greater than or equal to 500 microseconds, greater than or equal to 750 microseconds, greater than or equal to 1,000 microseconds, or greater than or equal to 4,000 microseconds and/or less than or equal to 1,000 microseconds, less than or equal to 800 microseconds, less than or equal to 600 microseconds, less than or equal to 150 microseconds, or less than or equal to 20 microseconds.

Other illustrative parameter for electrical stimulation delivered to the preganglionic dorsal root fibers may include, for example, synchronization (e.g., with multiple leads, electrodes, or electrode segments), or an on/off cycle to prevent battery drain and account for a wearing off time.

When preganglionic dorsal root fiber stimulation is used to reduce blood pressure in a patient with hypertension, the stimulation parameters may at least start with the following settings and be adjusted as needed. Both positive and negative electrodes may be used, and the electrodes may be guided by x-ray or ultrasound guidance to the preganglionic dorsal root fiber locations at T10-L1. The parameters may include, for example, amplitude (e.g., greater than or equal to 0.05 volts, greater than or equal to 0.15 volts, greater than or equal to 0.25 volts, greater than or equal to 0.5 volts, greater than or equal to 0.7 volts, or greater than or equal to 1 volt, and/or less than or equal to 1 volt, less than or equal to 0.8 volts, less than or equal to 0.45 volts, less than or equal to 0.2 volts, or less than or equal to 0.1 volts e.g., starting at 0.15 volts depending on skeletal muscle stimulation), frequency of the pulses (e.g., greater than or equal to 5 hertz, greater than or equal to 8 hertz, greater than or equal to 10 hertz, greater than or equal to 12 hertz, greater than or equal to 17 hertz, or greater than or equal to 25 hertz, and/or less than or equal to 30 hertz, less than or equal to 20 hertz, less than or equal to 15 hertz, less than or equal to 10 hertz, or less than or equal to 15 hertz, and/or between 10 hertz and 20 hertz, e.g., starting at 15 hertz), tonic or burst stimulation, pulse width of each pulse (e.g., greater than or equal to 20 microseconds, greater than or equal to 200 microseconds, greater than or equal to 350 microseconds, greater than or equal to 500 microseconds, greater than or equal to 750 microseconds, or greater than or equal to 1,000 microseconds, and/or less than or equal to 1,000 microseconds, less than or equal to 800 microseconds, less than or equal to 600 microseconds, less than or equal to 150 microseconds, or less than or equal to 20 microseconds, e.g., starting at 210 microseconds), and uni-or bi-lateral stimulation, e.g., starting on both sides of the spinal cord. An on/off cycle may be used, for example, to prevent battery drain, account for a wearing off time, or to maintain the effect of stimulation over time.

In one or more embodiments according to this disclosure, the shape and spread of a stimulation field may be controlled, for example, to adjust the fibers selected, manage stimulation response, or manage side effect response. In some embodiments, an electrode may be configured to deliver multi-polar stimulation, which may help localize stimulation. For example, an electrode may be configured as a tri-pole with a cathode and two anodes (+-+) (e.g., proximate to and spaced apart from the cathode or adjacent to the cathode) to contain the spread of the current flows from the cathode and fine tune the shape and spread of the stimulation field, such as to restrict stimulation of nerves other than the target nerves (e.g., the renal afferent nerves). Electrodes may additionally or alternatively include unbalanced multi-polar configurations. For example, an unbalanced tri-polar electrode may be configured with 75% of anodal current on a caudal electrode and 25% of anodal current on a cephalad electrode (i.e., 75%+, 100%−, 25%+). For another example, a multi-polar electrode may be configured with the cathodes unbalanced (e.g., 50%+, 25%−, 75%−, 50%+) to further fine tune the shape and spread of the stimulation field. In still another example, both the anodes and the cathodes may be unbalanced.

An illustrative method of preganglionic dorsal root fiber stimulation to activate a patient's reno-renal reflex, e.g., that may be performed or executed by the system of FIGS. 2-4, is depicted in FIG. 5A. When patients have congestive (or acute decompensated) heart failure 402, such patients may experience increased norepinephrine in the body, which reduces natriuresis/diuresis 410. Reduction in natriuresis/diuresis 410 may lead to administration of loop diuretics, which, in addition to the increase in norepinephrine, impairs the activation of the renal afferent nerves 406, which further suppresses the reno-renal reflexes.

As described herein, instead of administering loop diuretics to a patient, electrical stimulation may be delivered to the preganglionic dorsal root fibers of the T10 to L1 locations 408, as appropriate, to mimic the activation signals of the afferent nerves 406 to induce the reno-renal reflexes and restore, at least in part, the natural inhibition of the efferent renal nerve activity 404 to increase natriuresis/diuresis 410. In certain embodiments, physical parameters such as abdominal impedance may be measured or monitored 410 to determine whether or when preganglionic dorsal root fiber stimulation should be administered as discussed further below.

Activating the renal nerves (e.g., afferent nerves) through electrical stimulation of preganglionic dorsal root fibers to mimic the electrical activation signals of the renal nerves may also be used for other conditions, such as hypertension. When renal denervation is used to lower hypertension, a catheter is positioned in the renal arteries to ablate sympathetic nerves innervating the kidneys, which may lead to an increase in renal blood flow, an increase in urinary excretion of salt and water, and a decrease in renin release from the kidney along with other central sympathetic effects to reduce hypertension. However, problems associated with renal denervation include limited efficacy, limited applicability, and the possibility of unsustainability over time. For example, it is not a single nerve that needs to be denervated and the nerves are not always close to the vessel and may be closer to site branches. For another example, both renal afferent fibers and renal efferent fibers may be denervated, which may reduce efficacy in managing conditions such as hypertension. Also, exclusion criteria in two trials included >50% renal artery stenosis, eGFR<45 ml/min/1.73 m2, and renal artery anatomy that was unsuitable for ablation. Moreover, re-innervation has been shown to occur in rats after three months and sheep after eleven months.

As an alternative to using renal denervation to reduce hypertension, the preganglionic dorsal root fibers of the dorsal root(s) connected to the kidneys can be stimulated at the T10-L1 levels. The preganglionic dorsal root fibers stimulation may also increase renal blood flow, increase urinary excretion of salt and water, and decrease renin release from the kidney along with other central sympathetic effects to reduce hypertension. For example, a central sympathetic decrease would include a decrease in arterial and artery resistance decreasing afterload. Also, heart rate would be affected decreasing energy expenditure of the heart. Such stimulation may avoid incomplete targeting of nerves causing limited efficacy or concerns with re-innervation since nerves are not ablated with preganglionic dorsal root fiber stimulation. Instead, the reflex sending renal sensory information to the brain is interrupted, which in turn, affects kidney function. Using preganglionic dorsal root fiber stimulation for hypertension may also be administered to patients with arterial renal stenosis.

A block diagram of an illustrative method 450 of preganglionic dorsal root fiber stimulation to influence a decrease in blood pressure in patients with hypertension is shown in FIG. 5B. When a patient has hypertension, the patient may experience increased norepinephrine in the body which activates efferent renal nerves such as motor fibers and which reduces natriuresis/diuresis. Instead of renal denervation, electrical stimulation may be delivered to a patient's preganglionic dorsal root fibers of the T10 to L1 locations 408, as appropriate, to mimic the activation signals of the afferent nerves 412. This induces the reno-renal reflexes and restores, at least in part, the natural inhibition of the efferent renal nerve activity 416 to increase natriuresis/diuresis 418. Activation of the afferent nerves also decreases sympathetic output leading to a decrease in renin release, a decrease in vascular resistance, a decrease in afterload, and a decrease in heart rate, leading to less energy expenditure by the heart 414. Both the decrease in sympathetic output and increase in diuresis influence a decrease in blood pressure 420, thereby lowering a patient's hypertension. In certain embodiments, physical parameters such as blood pressure or heart rate may be measured or monitored to determine when preganglionic dorsal root fiber stimulation should be administered.

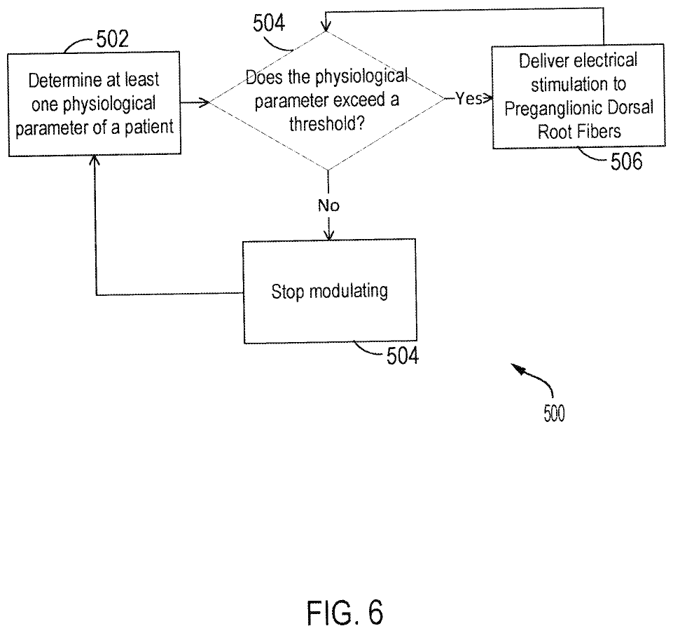

A block diagram of an illustrative method 500 of stimulating preganglionic dorsal root fibers in response to one or more physiological parameters is shown in FIG. 6. At least one physical parameter of a patient is determined (e.g., measured, monitored, determined from measurement data, etc.) 502. The at least one physiological parameter could include one, two, or more physiological, or physical, parameters such as, for example, a patient's creatinine level, blood urea nitrogen (BUN) level, respiration rate, abdominal fluid content, impedance in various regions of the body, ratios of impedance between body regions, pulmonary capillary wedge pressure, systolic blood pressure (SBP), diastolic blood pressure (DBP), brain natriuretic peptide (BNP) level, aortic blood pressure, venous blood pressure, brachial blood pressure, finger blood pressure, blood oxygen level (e.g., blood oxygen concentration), electrolyte level (e.g., electrolyte concentration), or rates of change thereof. One or more of the physical parameter values may be compared with a selected threshold value 504 that represents a point at which either preganglionic dorsal root fiber stimulation for a patient should be started (e.g., initiated, triggered, etc.), or in the case of ongoing preganglionic dorsal root fiber stimulation, the preganglionic dorsal root fiber stimulation should be adjusted (e.g., increasing stimulation parameters, decreasing stimulation parameters, terminating preganglionic dorsal root fiber stimulation, etc.).

Impedance may be measured using any suitable method. Suitable impedance measurement methods may include, for example, using an implantable medical device such as an implantable cardiac monitor (e.g., LINQ from MEDTRONIC), or using an external medical device such as an ankle band. Another suitable impedance measurement method includes measuring impedance in the epidural space, such as by measuring impedance between leads or electrodes positioned (e.g., implanted) in the epidural space. Yet another suitable impedance measurement method may be measuring the impedance between a lead positioned (e.g., implanted) in the epidural space and an implantable medical device case (e.g., control unit) positioned elsewhere in the patient's torso. Still another suitable measurement method includes measuring impedance between leads positioned in the epidural space (e.g., leads supporting electrodes for stimulation of preganglionic dorsal root fibers), an implantable medical device case positioned in the torso, and auxiliary leads supporting electrodes elsewhere in the torso. Still yet another suitable measurement method includes using jugular vein intra-or extra-vascular ultrasound to measure filling, corresponding to impedance.

If one or more of the physiological, or physical, parameters do not exceed a threshold, modulation is stopped 508. In this context, the term exceed means to go beyond the limits of, which in certain embodiments, may mean to increase above, or decrease below a threshold value. In certain embodiments, if a single parameter does not exceed a predetermined threshold, modulation may be stopped. In other embodiments where two or more parameters are determined, modulation may be stopped if only one, only two, only certain designated, or all parameters do not exceed one or more predetermined levels. The predetermined threshold may be determined for a designated demographic or personalized for each patient. For example, the selected threshold or parameters may be determined through a self-learning algorithm to improve, or optimize, conditions for initiating or altering preganglionic dorsal root fiber stimulation based on a subset of non-overlapping parameters. In certain embodiments one parameter (e.g., impedance) may inform how/when to stimulate based on a blood urea nitrogen (BUN) level, or vice versa. If it is determined that preganglionic dorsal root fiber stimulation does not need to be adjusted or started based on the comparison to selected threshold values, the method may return to measuring one or more physical parameters 502.

In an example, stimulation may be controlled based on selected thresholds for the patient's BUN level, such as based on whether the patient's BUN level is greater than a selected threshold. Selected thresholds for the patient's BUN level may include, for example, greater than or equal to 60 milligram per deciliter (mg/dL), greater than or equal to 70 mg/dL, greater than or equal to 75 mg/dL, greater than or equal to 80 mg/dL, greater than or equal to 90 mg/dL, or greater than or equal to 100 mg/dL, and/or less than or equal to 120 mg/dL, less than or equal to 105 mg/dL, less than or equal to 95 mg/dL, less than or equal to 85 mg/dL, or less than or equal to 75 mg/dL. In one embodiment, the selected threshold for the patient's BUN level may be 90 mg/dL.

In another example, stimulation additionally or alternatively may be controlled based on selected thresholds for the patient's creatine level, such as based on whether the patient's creatine level is greater than a selected threshold. Selected thresholds for the patient's creatine level may include, for example, greater than or equal to 0.8 milligram per deciliter (mg/dL), greater than or equal to 1 mg/dL, greater than or equal to 1.5 mg/dL, greater than or equal to 2 mg/dL, greater than or equal to 2.5 mg/dL, or greater than or equal to 3 mg/dL, and/or less than or equal to 3.5 mg/dL, less than or equal to 3 mg/dL, less than or equal to 2.75 mg/dL, less than or equal to 2.25 mg/dL, or less than or equal to 1.75 mg/dL. In one embodiment, the selected threshold for the patient's creatine level may be 2.75 mg/dL.

In yet another example, stimulation additionally or alternatively may be controlled based on selected thresholds for the patient's SBP, such as based on whether the patient's SBP is less than a selected threshold. Selected thresholds for the patient's SBP may include, for example, greater than or equal to 80 millimeters of mercury (mmHg), greater than or equal to 90 mmHg, greater than or equal to 100 mmHg, greater than or equal to 115 mmHg, greater than or equal to 125 mmHg, or greater than or equal to 140 mmHg, and/or less than or equal to 150 mmHg, less than or equal to 130 mmHg, less than or equal to 120 mmHg, less than or equal to 110 mmHg, or less than or equal to 100 mmHg. In one embodiment, the selected threshold for the patient's SBP may be 115 mmHg.

If the comparison to selected threshold values determines that preganglionic dorsal root fiber stimulation needs to be started or adjusted (e.g., a physical parameter equals or exceeds a selected threshold value), preganglionic dorsal root fiber stimulation is initiated (or adjusted) 506. As discussed previously, preganglionic dorsal root fiber stimulation may involve stimulating preganglionic dorsal root fibers of one dorsal root or of multiple dorsal roots at one or more levels of a patient's spine. In certain embodiments, if a single parameter exceeds a predetermined threshold, the preganglionic dorsal root fibers are stimulated. In other embodiments where two or more parameters are determined, stimulation is initiated if only one, only two, only certain designated, or all parameters exceed one or more predetermined levels. Stimulation is continued until one or more designated physical parameters no longer exceed the predetermined threshold(s). In certain embodiments the same physical parameter is used to initiated and halt preganglionic dorsal root fiber stimulation, and in other embodiments, different physical parameters may be used to start stimulation than are used to stop stimulation.

In an example, a single physical parameter such as a patient's BUN level may be measured and used to control preganglionic dorsal root fiber stimulation. The BUN level indicates the amount of nitrogen which originated from urine and is now present in the blood. BUN levels may be measured, for example, in blood using a chemical sensor based on a redox reaction. As BUN levels are higher in patients with heart failure, the BUN levels should decrease if diuresis increases and the blood urea nitrogen level can represent a level of diuresis. Thus, when a patient's BUN level is determined to be higher than a selected threshold, preganglionic dorsal root fiber stimulation is initiated at the T10-L1 region to activate renal afferent or other nerves innervating at least one of the patient's kidneys to increase diuresis as part of the reno-renal reflex. When the patient's BUN level no longer exceeds the selected threshold, preganglionic dorsal root fiber stimulation may be stopped.

In another example, two physical parameters are used together, such as a patient's BUN level and thoracic fluid content shift. A patient's thoracic fluid content shift can identify a measurement of a patient's abdominal impedance as well as an indirect measurement of a patient's subcutaneous impedance. For example, a patient's intra-abdominal pressure (IAP) is measured to provide an estimate of the amount of splanchnic bed overload, which at a certain point can develop a sudden decrease of capacitance and thus acute decompensated heart failure. The intra-abdominal pressure is determined by measuring the impedance of the abdominal region to represent congestion. Alternatively, the impedance of the lungs may be measured. Impedance can be measured using a technique disclosed in U.S. patent Application Publication No. 2018/0126172 entitled “Method and apparatus for monitoring tissue fluid content for use in an implantable cardiac device” and published May 10, 2018, which is incorporated herein by reference in its entirety. The impedance measurement is used to predict the amount of fluid that needs to be removed in acute heart failure syndromes like congestive heart failure.

The implantable system 300 of FIG. 4 may be used to measure or monitor both the BUN levels and the impedance of the patient's abdominal region. Alternatively, the BUN level may be measured offline by another technique. If the BUN level exceeds a selected threshold in combination with high impedance (e.g., impedance also exceeds a selected threshold), preganglionic dorsal root fiber stimulation is initiated at the T10-L1 region to induce diuresis and remove fluid from the body. In certain embodiments, if the BUN level exceeds the selected threshold, but the impedance, or thoracic fluid shift, has not, preganglionic dorsal root fiber stimulation is not initiated. In other embodiments, if the BUN level exceeds the selected threshold, but the impedance, or thoracic fluid shift, has not, preganglionic dorsal root fiber stimulation is initiated.

In some embodiments, the BUN level and thoracic fluid shift measurements are recorded regularly (e.g., every five minutes) with a lower frequency (e.g., every hour) during a time a patient is deemed to be sleeping. In addition, the timing and frequency of measurements can be adjusted in accordance with a patient's body position, physical activity, heart rate, or respiration measured with an on-board sensor. A patient's body position can influence the efficacy of the stimulation, and taking into account a patient's body position and activity level allows the device to decrease the frequency of measurements, alter the timing of stimulation, or adjust one or more stimulation parameters so as not to inconvenience the patient.

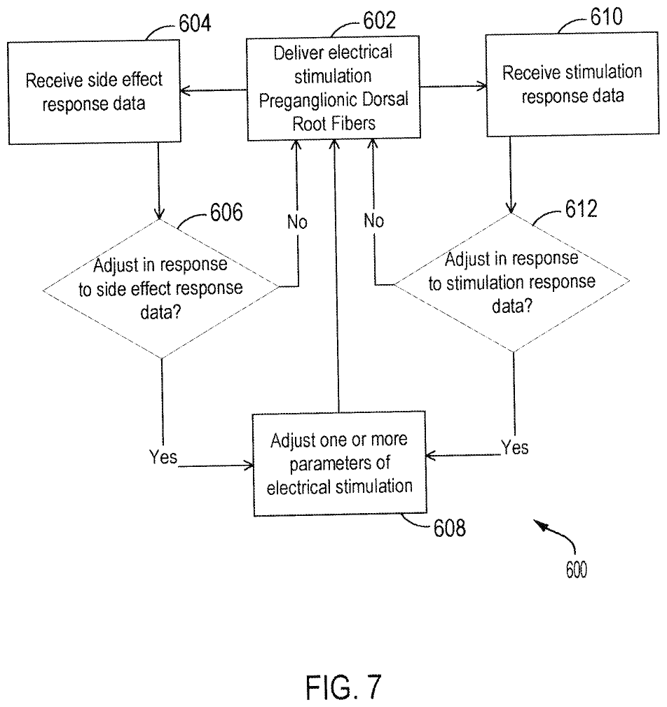

A block diagram of an illustrative method 600 of adjusting, or titrating, one or more parameters of electrical stimulation to preganglionic dorsal root fibers in response to data is illustrated in FIG. 7. Parameters of electrical stimulation to preganglionic dorsal root fibers may include any suitable parameters such as, for example, frequency, sinusoidal current, voltage, pulse width, tonic or burst stimulation, uni-or multi-lateral stimulation, balance of multi-lateral stimulation, on/off cycle timing. Electrical stimulation may be delivered using at least one electrode to preganglionic dorsal root fibers of at least one dorsal root 602, e.g., using the systems and devices described herein with respect to FIGS. 1-4.

The method 600 includes receiving data related to at least one side effect response 604. The side effect response data may include, for example, sensed data from the electrodes (e.g., EMG data or ECAP data), sensor measurements (e.g., electrolytes such as sodium, chloride, kalium, or creatinine), or patient feedback (e.g., reporting perceived side effects). Side effect response data may be related to side effects such as, for example, shivering, muscle contractions, pain, discomfort, dehydration (e.g., as indicated by electrolyte levels or impedance measurements), increased (i.e., elevated) heart rate, affected breathing (e.g., difficult breathing, such as due to muscle contractions), or sensory phenomenon (e.g., paresthesia).

Side effect response data may be used to determine whether to adjust one or more parameters 606 of the electrical stimulation delivered to the preganglionic dorsal root fibers. Side effect response data may indicate not to adjust stimulation parameters, for example, because all side effect responses are within an acceptable range (e.g., below a threshold perceived by the patient or below a hazard threshold). Side effect response data may indicate to adjust stimulation parameters, for example, because one or more side effect responses are outside of an acceptable range (e.g., above a threshold tolerated by the patient or above a hazard threshold). For example, an 5% to 10% increase in the patient's heart rate may be within the acceptable range of side effect responses. Additional examples of heart rate increases within a threshold of acceptable side effect responses may include, for example, greater than or equal to 1%, greater than or equal to 2.5%, greater than or equal to 4%, greater than or equal to 6%, greater than or equal to 9%, or greater than or equal to 12%, and/or less than or equal to 15%, less than or equal to 13%, less than or equal to 10%, less than or equal to 7%, or less than or equal to 5%. In one embodiment, the heart rate increase within a threshold of acceptable side effect responses may be 10%.

The acceptable range, or threshold, for side effect responses may differ based on circumstances, such as whether the patient is receiving stimulation to address acute symptoms (e.g., in a hospital) or chronic symptoms (e.g., chronic heart failure). For example, mild shivering may be within the acceptable range of side effect responses for a patient receiving stimulation to address acute symptoms, but mild shivering may be outside the acceptable range of side effect responses for a patient receiving stimulation chronically.

If side effect response data indicates to adjust stimulation parameters, one or more parameters of electrical stimulation may be adjusted 608. For example, if EMG data indicates motor response (e.g., muscle contractions or shivering) is above a perception threshold of the patient, one or more parameters of stimulation may be adjusted to reduce stimulation (e.g., by reducing sinusoidal current or voltage). For another example, if ECAP data indicates non-target fibers are being stimulated above an acceptable threshold (e.g., the patient's perception threshold), one or more parameters of stimulation (e.g., pulse width, amplitude, ratio of pulse width to amplitude to adjust sub-population of stimulated fibers based on the respective strength-duration curve) may be adjusted to reduce stimulation of the non-target fibers. For yet another example, if side effect response data indicates all side effect responses are well within acceptable ranges (e.g., EMG data indicates motor response is far below the patient's perception threshold), one or more stimulation parameters may be adjusted to increase, or up-titrate, stimulation (e.g., by increasing amplitude).

Further, the method 600 may further include receiving data related to at least one stimulation response 610. Stimulation response data may include, for example, sensed data from the electrodes (e.g., EMG data or ECAP data), sensor measurements (e.g., impedance, blood pressure), other measurements (e.g., measured urination) or patient feedback (e.g., increase urination experienced). Stimulation response data may be related to stimulation goals such as, for example, increasing diuresis, reducing fluid volume, reducing hypertension, reducing symptoms (e.g., swollen feet), improved breathing (e.g., less labored breathing), reduced pain, or reduced headache.

Stimulation response data is used to determine whether to adjust one or more parameters 612 of the electrical stimulation delivered to the preganglionic dorsal root fibers. Stimulation response data may indicate to not adjust stimulation parameters, for example, because all stimulation responses are within an acceptable range (e.g., diuresis is at or above a threshold effective to manage heart failure and is not causing dehydration). Stimulation response data may indicate to adjust stimulation parameters, for example, because one or more stimulation responses are outside of an acceptable range (e.g., diuresis is below a threshold effective to manage heart failure, blood pressure is too high, or thoracic volume is too high).

If stimulation response data indicates to adjust stimulation parameters, one or more parameters of electrical stimulation are adjusted 608. For example, if ECAP data indicates target fibers are being stimulated below an acceptable threshold (e.g., a threshold useful to stimulate the preganglionic dorsal root fibers to activate renal afferent nerves innervating at least one of the patient's kidneys to increase diuresis as part of the reno-renal reflex), one or more parameters of stimulation (e.g., pulse width, amplitude, ratio of pulse width to amplitude to adjust sub-population of stimulated fibers) may be adjusted to increase, or up-titrate, stimulation of target fibers. For another example, if stimulation response data indicates diuresis is below a threshold effective to manage heart failure, stimulation may be increased, such as by increasing amplitude.

Although the method 600 includes both adjusting one or more parameters of stimulation in response to side effect response data 606 and adjusting one or more parameters of stimulation in response to stimulation response data 612, the method may only include one of adjusting one or more parameters of stimulation in response to side effect response data 606 and adjusting one or more parameters of stimulation in response to stimulation response data 612.

### EXAMPLES

Example Ex1: A system comprising:
at least one electrode to deliver electrical stimulation to preganglionic dorsal root fibers of a patient to activate renal afferent nerves innervating at least one kidney of the patient; and
a computing apparatus comprising a processor and operably coupled to the at least one electrode, the computing apparatus is configured to control the electrical stimulation delivered using the at least one electrode to the preganglionic dorsal root fibers to inhibit activation of renal efferent nerves innervating the at least one kidney to promote diuresis.

Example Ex2: A method comprising:
delivering electrical stimulation using at least one electrode to preganglionic dorsal root fibers of a patient to activate renal afferent nerves innervating at least one kidney of the patient; and
controlling the electrical stimulation delivered using the at least one electrode to the preganglionic dorsal root fibers to inhibit activation of renal efferent nerves innervating the at least one kidney to promote diuresis.

Example Ex3: The system as in Example Ex1 or the method as in Example Ex2, wherein controlling the electrical stimulation delivered using the at least one electrode to the preganglionic dorsal root fibers comprises adjusting one or more parameters of the electrical stimulation delivered to the preganglionic dorsal root fibers using the at least one electrode, wherein the one or more parameters comprises one or more of pulse width, amplitude, frequency, on/off cycle timing, burst cycle timing, and pulse shape.

Example Ex4: The system or the method as in any one of Examples Ex1-Ex3, wherein the computing apparatus is further configured to execute or the method further comprises receiving electromyographic (EMG) data from the at least one electrode, and
wherein controlling the electrical stimulation delivered using the at least one electrode to the preganglionic dorsal root fibers comprises controlling the electrical stimulation delivered using the at least one electrode to the preganglionic dorsal root fibers based on the received EMG data.

Example Ex5: The system or the method as in any one of Examples Ex1-Ex4, wherein controlling the electrical stimulation delivered using the at least one electrode to the preganglionic dorsal root fibers comprises adjusting one or more parameters of the electrical stimulation delivered to the preganglionic dorsal root fibers using the at least one electrode in response to one or more of side effect response data and stimulation response data.

Example Ex6: The system or the method as in any one of Examples Ex1-Ex5, wherein the computing apparatus is further configured to execute or the method further comprises receiving evoked compound action potential (ECAP) signals from the at least one electrode in response to the delivery of electrical stimulation using the at least one electrode to the preganglionic dorsal root fiber, and
wherein adjusting one or more parameters of the electrical stimulation delivered to preganglionic dorsal root fibers using the at least one electrode comprises adjusting the one or more parameters of the electrical stimulation based on the received ECAP signals.

Example Ex7: The system or the method as in Example Ex6, wherein receiving ECAP signals from the at least one electrode in response to the delivery of electrical stimulation using the at least one electrode to the preganglionic dorsal root fiber comprises receiving ECAP signals from at least one sensing electrode in response to the delivery of electrical stimulation using the at least one electrode to the preganglionic dorsal root fiber.

Example Ex8: The system or the method as in one of Examples Ex6 or Ex7, wherein receiving ECAP signals from the at least one electrode in response to the delivery of electrical stimulation using the at least one electrode to the preganglionic dorsal root fiber comprises measuring a latency of response of the ECAP signals in response to the delivery of electrical stimulation using the at least one electrode to the preganglionic dorsal root fiber.

Example Ex9: The system or the method as in any one of Examples Ex1-Ex8, wherein controlling the electrical stimulation delivered using the at least one electrode to the preganglionic dorsal root fibers comprises adjusting a pulse width of the electrical stimulation delivered to the preganglionic dorsal root fibers or an amplitude of the electrical stimulation delivered to the preganglionic dorsal root fibers based on a strength-duration curve of the renal afferent nerves of the preganglionic dorsal root fiber.

Example Ex10: The system or the method as in any one of Examples Ex1-Ex9, wherein the at least one electrode comprises a cathode and an anode proximate to and spaced apart from the cathode, wherein controlling the electrical stimulation delivered using the at least one electrode to the preganglionic dorsal root fibers comprises delivering electrical stimulation using the cathode and using the anode to contain a cathodic current flow from the cathode to restrict stimulation of nerves other than the renal afferent nerves of the preganglionic dorsal root fiber.

Example Ex11: The system or the method as in any one of Examples Ex1-Ex10, wherein the at least one electrode comprises a multi-polar electrode configured as an unbalanced multi-polar electrode.

Example Ex12: The system or the method as in any one of Examples Ex1-Ex11, wherein the at least one electrode is configured to deliver stimulation to the preganglionic dorsal root fibers of at least one of the left or right dorsal root of at least one of the patient's T11 or T12 vertebrae.

Example Ex13: The system or the method as in any one of Examples Ex1-Ex12, wherein the at least one electrode comprises at least two electrodes to deliver electrical stimulation to the preganglionic dorsal root fibers of both the left and right dorsal root of at least one of the patient's T11 or T12 vertebrae.

Example Ex14: The system or the method as in any one of Examples Ex1-Ex13, wherein the at least one electrode comprises an implantable electrode to be implanted in the patient's body.

Example Ex15: The system or the method as in Example Ex14, wherein the implantable electrode is implanted between 2 millimeters and 6 millimeters from the patient's spinal midline.

Example Ex16: The system or the method as in any one of Examples Ex1-Ex15, wherein controlling the electrical stimulation delivered using the at least one electrode to the preganglionic dorsal root fibers comprises controlling the electrical stimulation to be delivered according to a predetermined schedule identifying one or more time periods when to deliver the electrical stimulation.

Example Ex17: The system or the method as in any one of Examples Ex1-Ex16, wherein controlling the electrical stimulation delivered using the at least one electrode to the preganglionic dorsal root fibers comprises controlling the electrical stimulation to be delivered based on the patient's circadian rhythm.

Example Ex18: The system or the method as in any one of Examples Ex 1-Ex17, wherein controlling the electrical stimulation delivered by the at least one electrode to the preganglionic dorsal root fibers comprises controlling the electrical stimulation to be delivered according to a user-defined schedule identifying one or more time periods when to deliver the electrical stimulation.

Example Ex19: The system or the method as in any one of Examples Ex1-Ex18, wherein controlling the electrical stimulation delivered by the at least one electrode to the preganglionic dorsal root fibers comprises controlling the electrical stimulation to be delivered based on an activity sensor.

Example Ex20: The system or the method as in any one of Examples Ex1-Ex19, wherein controlling the electrical stimulation delivered by the at least one electrode to the preganglionic dorsal root fibers comprises controlling the electrical stimulation to be delivered based on a sensed position of the patient's body.

Example Ex21: The system or the method as in any one of Examples Ex1-Ex20, wherein the system further comprises at least one sensor operably coupled to the computing apparatus to detect and the computing apparatus is further configured to execute or the method further comprises:
detecting using at least one sensor at least one physiological parameter of the patient, and
monitoring the at least one physiological parameter using the at least one sensor; and wherein controlling the electrical stimulation delivered by the at least one electrode to the preganglionic dorsal root fibers comprises controlling the electrical stimulation in response to the detected at least one physiological parameter.

Example Ex22: The system or the method as in Example Ex21, wherein the at least one physiological parameter of the patient includes one or more of a creatinine level, a urea nitrogen level, an electrolyte level, a blood urea nitrogen level, a respiration rate, a heart rate, a heart rate variability, an abdominal fluid content, a thoracic fluid content, a thoracic fluid content shift, a thoracic impedance, an abdominal impedance, an epidural impedance, a pulmonary arterial wedge pressure, and a capillary wedge pressure.

Example Ex23: The system or the method as in any one of Examples Ex21-Ex22, wherein the computing apparatus is further configured to execute or the method further comprises determining a rate of change of the monitored at least one physiological parameter, wherein controlling the electrical stimulation in response to the detected physiological parameter comprises controlling the electrical stimulation in response to the determined rate of change of the monitored at least one physiological parameter.

Example Ex24: The system or the method as in any one of Examples Ex21-Ex23, wherein controlling the electrical stimulation in response to the determined rate of change of the monitored at least one physiological parameter comprises adjusting the electrical stimulation in response to the determined rate of change of the monitored at least one physiological parameter being greater than or equal to a threshold.

Example Ex25: The system or the method as in any one of Examples Ex21-Ex24, wherein controlling the electrical stimulation in response to the detected physiological parameter comprises controlling the electrical stimulation delivered to the preganglionic dorsal root fibers using the at least one electrode in response to the detected physiological parameter being equal to or greater than a threshold.

Example Ex26: The system or the method as in any one of Examples Ex21-Ex25, wherein monitoring the at least one physiological parameter using the at least one sensor comprises monitoring a first physiological parameter and a second physiological parameter,
wherein controlling the electrical stimulation in response to the detected at least one physiological parameter comprises controlling the electrical stimulation in response to the first physiological parameter and the second physiological parameter.

Example Ex27: The system or the method as in Example Ex26, wherein the first physiological parameter is a blood urea nitrogen concentration and the second physiological parameter is an abdominal impedance.

What is claimed:

1. A system comprising:
at least one electrode to deliver electrical stimulation to preganglionic dorsal root fibers of a patient to activate renal afferent nerves innervating at least one kidney of the patient; and
a computing apparatus comprising a processor and operably coupled to the at least one electrode, the computing apparatus is configured to control the electrical stimulation delivered using the at least one electrode to the preganglionic dorsal root fibers to inhibit activation of renal efferent nerves innervating the at least one kidney to promote diuresis.

2. The system as in claim 1, wherein controlling the electrical stimulation delivered using the at least one electrode to the preganglionic dorsal root fibers comprises adjusting one or more parameters of the electrical stimulation delivered to the preganglionic dorsal root fibers using the at least one electrode, wherein the one or more parameters comprises one or more of pulse width, amplitude, frequency, on/off cycle timing, burst cycle timing, and pulse shape.

3. The system as in claim 1, wherein the computing apparatus is further configured to execute receiving electromyographic (EMG) data from the at least one electrode, and
wherein controlling the electrical stimulation delivered using the at least one electrode to the preganglionic dorsal root fibers comprises controlling the electrical stimulation delivered using the at least one electrode to the preganglionic dorsal root fibers based on the received EMG data.

4. The system of claim 1, wherein controlling the electrical stimulation delivered using the at least one electrode to the preganglionic dorsal root fibers comprises adjusting one or more parameters of the electrical stimulation delivered to the preganglionic dorsal root fibers using the at least one electrode in response to one or more of side effect response data and stimulation response data.

5. The system of claim 4, wherein the computing apparatus is further configured to execute receiving evoked compound action potential (ECAP) signals from the at least one electrode in response to the delivery of electrical stimulation using the at least one electrode to the preganglionic dorsal root fiber, and
wherein adjusting one or more parameters of the electrical stimulation delivered to preganglionic dorsal root fibers using the at least one electrode comprises adjusting the one or more parameters of the electrical stimulation based on the received ECAP signals.

6. The system of claim 5, wherein receiving ECAP signals from the at least one electrode in response to the delivery of electrical stimulation using the at least one electrode to the preganglionic dorsal root fiber comprises receiving ECAP signals from at least one sensing electrode in response to the delivery of electrical stimulation using the at least one electrode to the preganglionic dorsal root fiber.

7. The system of claim 5, wherein receiving ECAP signals from the at least one electrode in response to the delivery of electrical stimulation using the at least one electrode to the preganglionic dorsal root fiber comprises measuring a latency of response of the ECAP signals in response to the delivery of electrical stimulation using the at least one electrode to the preganglionic dorsal root fiber.

8. The system of claim 1, wherein controlling the electrical stimulation delivered using the at least one electrode to the preganglionic dorsal root fibers comprises adjusting a pulse width of the electrical stimulation delivered to the preganglionic dorsal root fibers or an amplitude of the electrical stimulation delivered to the preganglionic dorsal root fibers based on a strength-duration curve of the renal afferent nerves of the preganglionic dorsal root fiber.

9. The system of claim 1, wherein the at least one electrode comprises a cathode and an anode proximate to and spaced apart from the cathode, wherein controlling the electrical stimulation delivered using the at least one electrode to the preganglionic dorsal root fibers comprises delivering electrical stimulation using the cathode and using the anode to contain a cathodic current flow from the cathode to restrict stimulation of nerves other than the renal afferent nerves of the preganglionic dorsal root fiber.

10. The system of claim 1, wherein the at least one electrode comprises a multi-polar electrode configured as an unbalanced multi-polar electrode.

11. The system of claim 1, wherein the at least one electrode is configured to deliver stimulation to the preganglionic dorsal root fibers of at least one of the left or right dorsal root of at least one of the patient's T11 or T12 vertebrae.

12. The system of claim 1, wherein the at least one electrode comprises at least two electrodes to deliver electrical stimulation to the preganglionic dorsal root fibers of both the left and right dorsal root of at least one of the patient's T11 or T12 vertebrae.

13. The system of claim 1, wherein the at least one electrode comprises an implantable electrode to be implanted in the patient's body.

14. The system of claim 13, wherein the implantable electrode is configured to be implanted between 2 millimeters and 6 millimeters from the patient's spinal midline.

15. The system of claim 1, wherein the electrical stimulation delivered using the at least one electrode to the preganglionic dorsal root fibers comprises controlling the electrical stimulation to be delivered according to a predetermined schedule identifying one or more time periods when to deliver the electrical stimulation.

16. The system of claim 1, wherein controlling the electrical stimulation delivered using the at least one electrode to the preganglionic dorsal root fibers comprises controlling the electrical stimulation to be delivered based on the patient's circadian rhythm.

17. The system of claim 1, wherein controlling the electrical stimulation delivered by the at least one electrode to the preganglionic dorsal root fibers comprises controlling the electrical stimulation to be delivered based on an activity sensor.

18. The system of claim 1, wherein controlling the electrical stimulation delivered by the at least one electrode to the preganglionic dorsal root fibers comprises controlling the electrical stimulation to be delivered based on a sensed position of the patient's body.

19. The system of claim 1, wherein the system further comprises at least one sensor operably coupled to the computing apparatus to detect and the computing apparatus is further configured to execute
detecting using at least one sensor at least one physiological parameter of the patient, and
monitoring the at least one physiological parameter using the at least one sensor; and
wherein controlling the electrical stimulation delivered by the at least one electrode to the preganglionic dorsal root fibers comprises controlling the electrical stimulation in response to the detected at least one physiological parameter.

20. The system of claim 19, wherein the at least one physiological parameter of the patient includes one or more of a creatinine level, a urea nitrogen level, an electrolyte level, a blood urea nitrogen level, a respiration rate, a heart rate, a heart rate variability, an abdominal fluid content, a thoracic fluid content, a thoracic fluid content shift, a thoracic impedance, an abdominal impedance, an epidural impedance, a pulmonary arterial wedge pressure, and a capillary wedge pressure.

21. The system of claim 19, wherein the computing apparatus is further configured to execute determining a rate of change of the monitored at least one physiological parameter, wherein controlling the electrical stimulation in response to the detected physiological parameter comprises controlling the electrical stimulation in response to the determined rate of change of the monitored at least one physiological parameter.

22. The system of claim 19, wherein monitoring the at least one physiological parameter using the at least one sensor comprises monitoring a first physiological parameter and a second physiological parameter,
wherein controlling the electrical stimulation in response to the detected at least one physiological parameter comprises controlling the electrical stimulation in response to the first physiological parameter and the second physiological parameter.

23. The system as in claim 22, wherein the first physiological parameter is a blood urea nitrogen concentration and the second physiological parameter is an abdominal impedance.

24. A method comprising:
Delivering electrical stimulation using at least one electrode to preganglionic dorsal root fibers of a patient to activate renal afferent nerves innervating at least one kidney of the patient; and
controlling the electrical stimulation delivered using the at least one electrode to the preganglionic dorsal root fibers to inhibit activation of renal efferent nerves innervating the at least one kidney to promote diuresis.
